(19) United States
(12) Patent Application Publication
(10) Pub. No.: US 2026/0355557 A1
(43) Pub. Date: Nov. 12, 2026

(54) FIRE PREVENTION DEVICE FOR MULTIPARAMETER-BASED BATTERY CELL AND METHOD THEREOF

(71) Applicant: evParts Co., Ltd., Goyang-si (KR)

(72) Inventor: Tae-Hoon KIM, Seoul (KR)

(21) Appl. No.: 19/406,169

(22) Filed: Dec. 2, 2025

(30) Foreign Application Priority Data
May 12, 2025 (KR) 10-2025-0060937

(51) Int. Cl. A62C 37/40; A62C 3/16; G01K 3/00; G01N 33/00; G01R 31/367; G01R 31/374; G01R 31/3835; G01R 31/389; H01M 10/42; H01M 10/48

(52) U.S. Cl. CPC A62C 37/40; A62C 3/16; G01K 3/005; G01N 33/0027; G01R 31/367; G01R 31/374; G01R 31/3835; G01R 31/389; H01M 10/425; H01M 10/486; H01M 2010/4271

(57) ABSTRACT

An embodiment relates to a fire prevention device for a multiparameter-based battery cell and a method thereof, in which a good/bad determination is made on the basis of parameters of any two or more of cell voltage, temperature, internal resistance, released gas, and internal pressure value of a portable auxiliary battery in order to prevent a fire that may occur in the portable auxiliary battery.

### CROSS REFERENCE TO RELATED APPLICATION

The present application claims priority to Korean Patent Application No. 10-2025-0060937, filed on May 12, 2025, the entire contents of which are incorporated herein for all purposes by this reference.

### BACKGROUND OF THE INVENTION

### Field of the Invention

The present disclosure relates to a fire prevention device for a multiparameter-based battery cell and a method thereof and, more particularly, to a fire prevention device for a multiparameter-based battery cell and a method thereof, in which a good/bad determination is made on the basis of parameters of any two or more of cell voltage, temperature, internal resistance, released gas, and internal pressure value of a portable auxiliary battery in order to prevent a fire that may occur in the portable auxiliary battery.

### Description of the Related Art

Portable auxiliary batteries are devices each having a built-in large-capacity lithium-ion battery, thereby charging other devices such as a smartphone or being used as an emergency power source. For example, in the event of a fire, these portable auxiliary batteries have a problem in that high-temperature flames are generated instantly due to thermal runaway of the lithium-ion battery, and the extinguishing of the fire requires a lot of time, manpower, money, etc. In particular, if a fire breaks out in a portable auxiliary battery carried onto an aircraft, the fire may cause significant material damage as well as death and injury of passengers.

To prevent such a problem, various regulations are being implemented, such as prohibiting the storage of portable auxiliary batteries on aircraft shelves and taking measures to prevent short circuits of the portable auxiliary batteries. The measures for preventing short circuits of the portable auxiliary batteries include a method of storing each battery separately in a zipper bag, a method of storing these batteries in individual protective pouches, a method of attaching insulating tape to each battery terminal, etc. In addition, portable auxiliary batteries with a capacity exceeding a predetermined level of 160 Wh and the like are prohibited from being brought onto aircraft. However, the above-described regulations are only temporary measures and have limitations in fundamentally preventing fire outbreak in the portable auxiliary batteries.

Methods of detecting fire risk of a portable auxiliary battery include a first method of checking the condition of the portable auxiliary battery through electrical sensing and a second method of checking internal structural deformation of the portable auxiliary battery by using an ultrasonic sensor or X-ray.

The first method described above determines the safety of the portable auxiliary battery on the basis of a single parameter such as voltage or current, but it is practically impossible to determine all types of portable auxiliary batteries by using only a single voltage value or a single current value. The second method is only able to determine structural deformation or cracks inside the portable auxiliary battery, and is not an optimized method for determining the possibility of fire.

In order to solve the problems described above, the inventor of the embodiment of the present disclosure proposes a novel fire prevention device for a multiparameter-based battery cell and a method thereof, and the details of which will be described below.

### Documents of Related Art

(Patent Document 1) Korean Patent Application Publication No. 10-2025-0054847 “BATTERY MODULE MANUFACTURING DEVICE CAPABLE OF MINIMIZING DEFECTS AND BATTERY MANAGEMENT SYSTEM MANUFACTURED THEREBY”

### SUMMARY OF THE INVENTION

Proposed is devised to solve the problems of the related art mentioned above.

An objective of the present disclosure is to provide a fire prevention device for a multiparameter-based battery cell and a method thereof, in which a good/bad determination is made on the basis of parameters of any two or more of cell voltage, temperature, internal resistance, released gas, and internal pressure value of a portable auxiliary battery, so as to prevent a fire that may occur in the portable auxiliary battery.

In addition, another objective of the present disclosure is to provide a fire prevention device for a multiparameter-based battery cell and a method thereof, in which airport security personnel and others are enabled to relatively make an easy determination on the safety of a plurality of portable auxiliary batteries by printing a specific pattern containing information of a good/bad determination on a printed material attachable to each portable auxiliary battery after making the good/bad determination on the portable auxiliary battery.

In order to achieve the above-described objectives, the present disclosure can be realized by the exemplary embodiments having the following configuration.

According to one exemplary embodiment of the present disclosure, there is provided a fire prevention device for a multiparameter-based battery cell, the fire prevention device including: a connector connected to a charging port of a portable auxiliary battery; a discharger for discharging the battery cell of the portable auxiliary battery through the connector; an impedance measurement device for measuring internal impedance of the portable auxiliary battery connected through the connector; a controller configured to obtain BMS information of the portable auxiliary battery and make a determination of whether the portable auxiliary battery is in good or bad condition on the basis of a plurality of parameters of voltage, resistance, and impedance; and a database.

According to another exemplary embodiment of the present disclosure, the discharger may include a resistance-value measurement module configured to measure a direct current (DC) internal resistance value of the portable auxiliary battery.

According to yet another exemplary embodiment of the present disclosure, the controller may obtain a first temperature of the battery cell of the portable auxiliary battery measured through a first temperature sensor mounted on the portable auxiliary battery and a second temperature, which is an indoor temperature, measured through a second temperature sensor installed on one side of the fire prevention device, and
the controller may include a temperature-based determination module configured to make the determination of whether the portable auxiliary battery is in the good or bad condition by comparing the first temperature and the second temperature.

According to still another exemplary embodiment of the present disclosure, the controller may include a voltage-based determination module configured to make the determination of whether the portable auxiliary battery is in the good or bad condition by comparing a voltage value measured at an arbitrary State Of Charge (SOC) of the portable auxiliary battery discharged by the discharger with a voltage value for each SOC, stored in the database, of the portable auxiliary battery in normal condition.

According to still another exemplary embodiment of the present disclosure, the controller may include a resistance-based determination module configured to make the determination of whether the portable auxiliary battery is in the good or bad condition through the DC internal resistance value of the portable auxiliary battery measured by the resistance-value measurement module.

According to still another exemplary embodiment of the present disclosure, the resistance-based determination module may make the determination of whether the portable auxiliary battery is in the good or bad condition through a first resistance value and a second resistance value, which are the portable auxiliary battery's DC internal resistance values for respective SOCs different from each other.

According to still another exemplary embodiment of the present disclosure, the controller may include an impedance-based determination module configured to make the determination of whether the portable auxiliary battery is in the good or bad condition by comparing impedance measured at an arbitrary SOC of the portable auxiliary battery discharged by the discharger with impedance for each SOC, stored in the database, of the portable auxiliary battery in normal condition.

According to still another exemplary embodiment of the present disclosure, the controller may include a battery-condition score calculation module configured to calculate a condition score of the portable auxiliary battery on the basis of a temperature of the portable auxiliary battery and a difference between impedance at a specific SOC of the portable auxiliary battery and impedance at a corresponding SOC stored in the database.

According to still another exemplary embodiment of the present disclosure, the fire prevention device may further include a printing module configured to print a barcode or a QR code containing information of the good/bad determination on a printed material to be provided to a user after making the determination of whether the portable auxiliary battery is in the good or bad condition through the controller.

According to still another exemplary embodiment of the present disclosure, the fire prevention device may further include an airtight part configured to allow the portable auxiliary battery connected to the connector to be received.

According to still another exemplary embodiment of the present disclosure, the airtight part may include a gas detection sensor installed on one side thereof and configured to detect gas released from the portable auxiliary battery into an internal space of the airtight part.

According to still another exemplary embodiment of the present disclosure, the airtight part may further include a pressure sensor installed on one side thereof and configured to detect a pressure value during a discharging process of the portable auxiliary battery.

According to still another exemplary embodiment of the present disclosure, the database may store information of the good/bad determination of the portable auxiliary battery, the information being determined by the controller and matched with a passenger's boarding pass information.

According to still another exemplary embodiment of the present disclosure, the fire prevention device may further include a housing part forming an exterior of the fire prevention device; and a boarding pass reader installed on one side of the housing part and to which a boarding pass for each passenger is allowed to be inserted.

According to still another exemplary embodiment of the present disclosure, the airtight part may further include an automatic fire extinguishing part configured to automatically extinguish a fire occurring in the internal space of the airtight part.

According to still another exemplary embodiment of the present disclosure, the airtight part may further include a gas suction part configured to allow gas released outside a casing of the portable auxiliary battery during a charging and discharging process of the portable auxiliary battery to be released outside the airtight part.

The embodiment of the present disclosure has the following effects through the above-mentioned configuration.

The embodiment of the present disclosure has an effect that a good/bad determination is made on the basis of parameters of any two or more of cell voltage, temperature, internal resistance, released gas, and internal pressure value of a portable auxiliary battery, so as to prevent a fire that may occur in the portable auxiliary battery.

In addition, the embodiment of the present disclosure has another effect of enabling airport security personnel and others to relatively make an easy determination on the safety of a plurality of portable auxiliary batteries by printing a specific pattern containing information of a good/bad determination on a printed material attachable to each portable auxiliary battery after making a good/bad determination on the portable auxiliary battery.

Meanwhile, even though effects are not explicitly mentioned herein, it is affirmed that effects described in the following specification and potential effects thereof, the effects being expected by the technical features of the present disclosure, are treated as the same as described in the specification of the present disclosure.

### BRIEF DESCRIPTION OF THE DRAWINGS

FIG. 1 is a reference view of a fire prevention device for a multiparameter-based battery cell according to one exemplary embodiment of the present disclosure.

FIG. 2 is a block view of the fire prevention device for the multiparameter-based battery cell according to FIG. 1.

FIG. 3 is a reference view illustrating an airtight part according to FIG. 2.

FIG. 4 is a block view of a controller according to FIG. 2.

FIG. 5 is a flowchart of a fire prevention method for a multiparameter-based battery cell according to one exemplary embodiment of the present disclosure.

FIG. 6 is a flowchart of step S40 according to FIG. 5.

FIG. 7 is a flowchart of step S50 according to FIG. 5.

FIG. 8 is a flowchart of step S60 according to FIG. 5.

FIG. 9 is a flowchart of step S70 according to FIG. 5.

### DETAILED DESCRIPTION OF THE INVENTION

Hereinafter, exemplary embodiments of the present disclosure will be described in detail with reference to the accompanying drawings. The exemplary embodiments of the disclosure may be modified in various forms, and the scope of the present disclosure should not be construed as being limited to the following exemplary embodiments, but should be interpreted on the basis of the matters described in the claims. In addition, the present exemplary embodiments are only provided for reference in order to more completely describe the present disclosure to those skilled in the art.

As used herein, expression in the singular forms may include expression in the plural forms, unless the context clearly indicates otherwise. In addition, as used herein, “comprise” and/or “comprising” specify the presence of the recited shapes, numbers, steps, operations, members, elements, and/or groups thereof, and does not exclude the presence or addition of one or more other shapes, numbers, operations, members, elements and/or groups thereof.

In addition, below, there are some components that are described as “first”, “second”, etc., which are indicated with numbers, and in this case, it should be noted that a second component does not presuppose a first component, and each component is independent of each other.

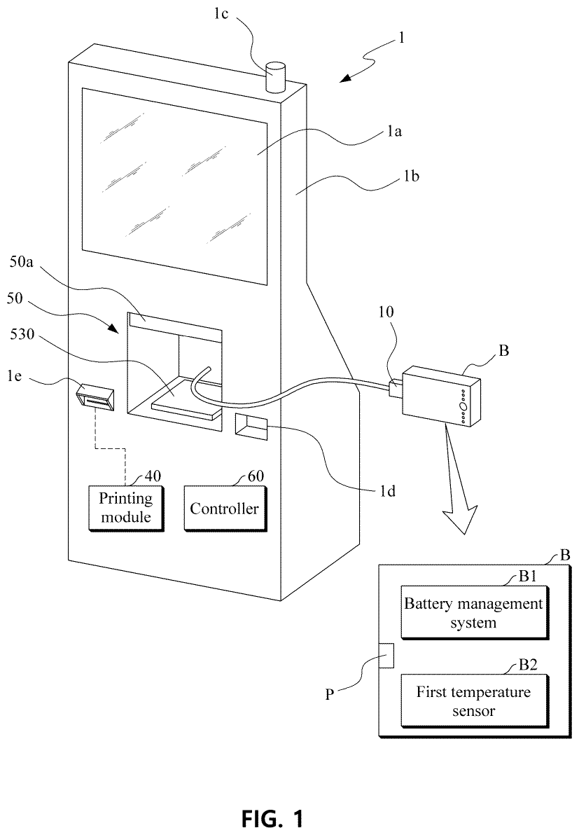

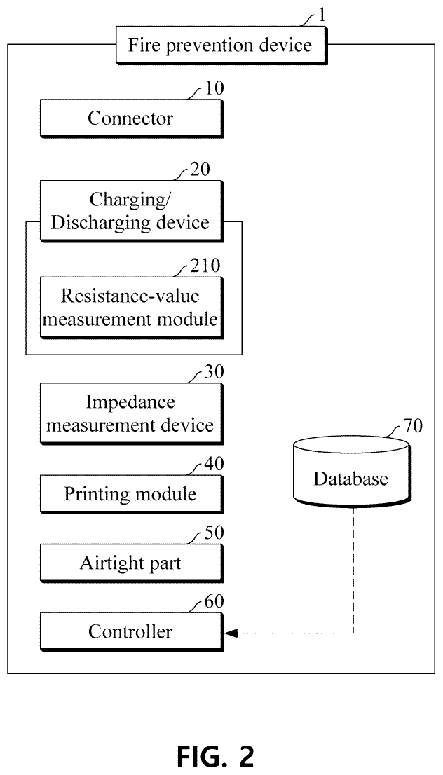

FIG. 1 is a reference view of a fire prevention device for a multiparameter-based battery cell according to one exemplary embodiment of the present disclosure, and FIG. 2 is a block view of the fire prevention device for the multiparameter-based battery cell according to FIG. 1.

Hereinafter, the fire prevention device for the multiparameter-based battery cell according to one exemplary embodiment of the present disclosure will be described in detail with reference to the attached drawings.

The above “battery cell” is understood to refer to a portable auxiliary battery for a smartphone (hereinafter referred to as “portable auxiliary battery”).

Referring to FIGS. 1 and 2, the present disclosure relates to a fire prevention device 1 for a multiparameter-based battery cell and a method S1 thereof and, more particularly, to a fire prevention device 1 for a multiparameter-based battery cell and a method S1 thereof, in which a good/bad determination is made on the basis of parameters of any two or more of cell voltage, temperature, internal resistance, released gas, and internal pressure value of a portable auxiliary battery B in order to prevent a fire that may occur in the portable auxiliary battery B.

According to one exemplary embodiment of the present disclosure, for example, the fire prevention devices 1 is proposed to make a good/bad determination on a portable auxiliary battery B owned by a passenger scheduled to board an aircraft, and it is preferable that at least one or more of the fire prevention devices 1 are installed in an airport.

To this end, the fire prevention device 1 may include a connector 10, a charging/discharging device 20, an impedance measurement device 30, a printing module 40, an airtight part 50, a controller 60, and a database 70.

The connector 10 is a component arranged on one side of the fire prevention device 1 and configured to be connected to the portable auxiliary battery B through a charging port P of the portable auxiliary battery B. Accordingly, BMS information of the portable auxiliary battery B may be obtained through the connector 10 and communication interface. In this case, the BMS information may be obtained by the controller 60. The term “BMS information” is preferably one or more kinds of information selected from among State Of Charge (SOC) information, cell voltage, cell temperature, internal resistance values, and the like of the portable auxiliary battery B, and is more preferable to be information including all kinds of the above information. To this end, a battery management system B1 is built into the portable auxiliary battery B, and the fire prevention device 1 may obtain the BMS information from the battery management system B1. In addition, a first temperature sensor B2 may be installed on an inner side a casing of the portable auxiliary battery B so as to measure a temperature value of a battery cell. A second temperature sensor 1c is also installed on one side of a housing part 1b of the fire prevention device 1, so that an indoor temperature at an airport where the fire prevention device 1 is arranged may be measured. Additionally, a user display 1a may be further provided on the front side of the housing part 1b.

The charging/discharging device 20 is a component for charging and/or discharging the battery cell of the portable auxiliary battery B connected to the fire prevention device 1 by the connector 10. Such a charging/discharging device 20 is able to maintain the battery cell in a fully charged state, a fully discharged state, or a partially charged state. In this case, as described above, it should be noted that the charging/discharging device 20 may be a component for charging and discharging the battery cell of the portable auxiliary battery or may also be a component as a discharger only performing a discharging operation on the battery cell. Accordingly, the charging/discharging device 20 may be referred to as a discharger depending on circumstances. The control of charging and/or discharging of the battery cell of the portable auxiliary battery B through the charging/discharging device 20 may be performed through the controller 60 described below later.

In addition, the charging/discharging device 20 may include a resistance-value measurement module 210.

The resistance-value measurement module 210 is a module for measuring a resistance value R of the portable auxiliary battery B. For example, by using the charging/discharging device 20 under the control of the controller 60, the battery cell of the portable auxiliary battery B is charged/discharged at a specific current rate I for a predetermined period of time, so that a voltage V1 immediately before charging/discharging and a voltage V2 after the charging/discharging are recorded. At this time, the resistance-value measurement module 210 may derive the resistance value R by Equation (1). That is, the resistance-value measurement module 210 may measure a direct current (DC) internal resistance value of the portable auxiliary battery B.

R=(V2−V1)/I (1)

The impedance measurement device 30 is a component for measuring impedance of the portable auxiliary battery B connected to the fire prevention device 1 by the connector 10. For example, the impedance of the battery cell may be measured by applying a sinusoidal alternating current (AC) to the battery cell of the portable auxiliary battery B through the impedance measurement device 30 under the control of the controller 60 and then specifying voltage response corresponding thereto. Accordingly, the AC internal impedance of the portable auxiliary battery B may be measured by the impedance measurement device 30. In this case, the voltage response may be measured at a specific frequency of, for example, 1 kHz, but the scope of the present disclosure is not limited by this numerical range.

The printing module 40 is a module for printing a specific pattern containing information of good/bad determination on a printed material after making a determination of whether the portable auxiliary battery B connected to the connector 10 is in good or bad condition through the controller 60 described below later. The specific pattern above may be a barcode or a QR code. In addition, for the portable auxiliary battery B, the specific pattern may include the information of good/bad determination, inspection dates and time, a passenger, and a test measurement value (e.g., information about the cell voltage, temperature, internal resistance of the portable auxiliary battery B). In addition, the printed material may be a sticker with the barcode or QR code printed thereon, and a passenger who owns the portable auxiliary battery B can attach the printed material to his or her portable auxiliary battery B. In addition, on one side of the housing part 1b of the fire prevention device 1, a printout provision part 1e may be formed to allow the printed material produced by the printing module 40 to be outputted and provided to the outside. In such a way, the printed material produced by the printing module 40 is able to be attached to the portable auxiliary battery B, so the airport security personnel and others may relatively easily make a safety determination on a plurality of portable auxiliary batteries B.

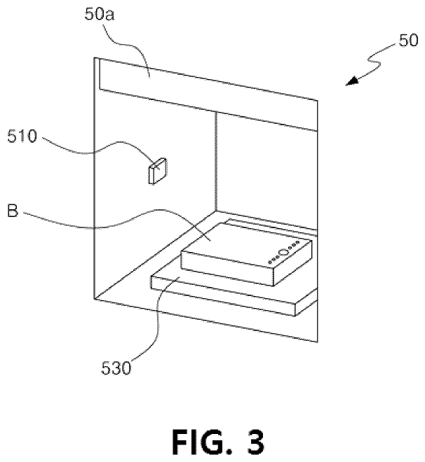

FIG. 3 is a reference view illustrating an airtight part according to FIG. 2.

Referring to FIGS. 1 to 3, the airtight part 50 is a component for receiving the portable auxiliary battery B connected to the connector 10. Such an airtight part 50 is formed on another side of the housing part 1b of the fire prevention device 1, so as to enable the portable auxiliary battery B received in the internal space of the airtight part 50 to be blocked from the external space. For example, the airtight part 50 may be a component to be a chamber. To this end, a door 50a for opening and closing the internal space may also be installed in the airtight part 50. For example, the door 50a may slide in an up-and-down direction to open and close the airtight part 50, but the scope of the present disclosure is not limited thereto.

In addition, the airtight part 50 may include a gas detection sensor 510 and a pressure sensor 530 in the internal space thereof.

The gas detection sensor 510 is a component as a sensor installed on one side of the airtight part 50 and configured to detect gas released outside the casing of the portable auxiliary battery B during the charging and discharging process of the portable auxiliary battery B by the charging/discharging device 20. In this case, the gas detection sensor 510 may detect one or more gases from among CO₂, CO, H₂, and H₄, which are released into the internal space of the airtight part 50. A value of output detected by the gas detection sensor 510 may be transmitted to the controller 60.

The pressure sensor 530 is a component as a sensor installed on yet another side of the airtight part 50 to detect a pressure value of the portable auxiliary battery B during the charging and discharging process of the portable auxiliary battery B by the charging/discharging device 20. A value of output detected by the pressure sensor 530 may be transmitted to the controller 60.

In addition, it is preferable that the airtight part 50 has an explosion-proof structure, and further includes an automatic fire extinguishing part (not shown) on still another side thereof. For example, there is a possibility that a fire may occur in the internal space of the airtight part 50 when gas is released from the portable auxiliary battery B or excessive swelling occurs in the portable auxiliary battery B. To prevent this kind of fire, it is preferable that the airtight part 50 has the automatic fire extinguishing part on still another side thereof. The automatic fire extinguishing part may have an automatic class D fire extinguishing device (or class D fire extinguisher).

In addition, it is preferable that the airtight part 50 further includes a gas suction part (not shown) on further another side thereof. In this way, the gas released outside the casing of the portable auxiliary battery B during the charging and discharging process of the portable auxiliary battery B may be released outside the airtight part 50 by the gas suction unit, having no gas left in the internal space of the airtight part 50. In addition, it is more preferable that the gas suction unit includes a filter structure on one side thereof, thereby capturing the gas.

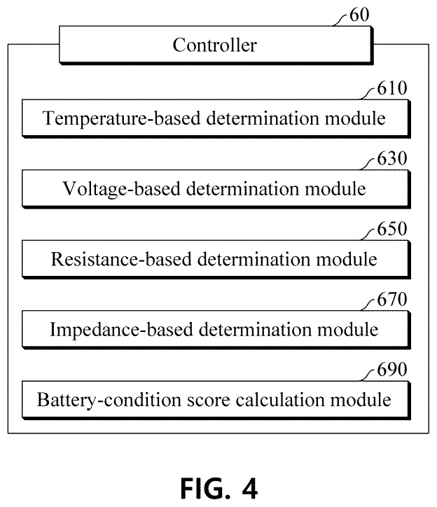

FIG. 4 is a block view of a controller according to FIG. 2.

Referring to FIGS. 1, 2, and 4, the controller 60 is a component for controlling all the components of the fire prevention device 1. In addition, the controller 60 may make a determination of whether the portable auxiliary battery B connected to the connector 10 is in good or bad condition. At this time, the portable power battery B being bad may mean that there is a risk of fire with the portable power battery B, making it impossible to bring the portable power battery B onto an aircraft.

To this end, the controller 60 may include a temperature-based determination module 610, a voltage-based determination module 630, a resistance-based determination module 650, an impedance-based determination module 670, and a battery-condition score calculation module 690.

The temperature-based determination module 610 is a module configured to make a determination of whether the portable auxiliary battery B is in good or bad condition by comparing a battery cell temperature of the portable auxiliary battery B measured by the first temperature sensor B2 and an airport indoor temperature measured by the second temperature sensor 1c. For example, the temperature-based determination module 610 determines that the portable auxiliary battery B is bad when the temperature of the battery cell exceeds a preset percentage based on an indoor temperature and/or when the temperature of the battery cell exceeds a preset value. For example, the temperature-based determination module 610 may determine that the portable auxiliary battery B is bad when the temperature of the battery cell exceeds 20% of the indoor temperature and/or when the temperature of the battery cell exceeds 40 °C.

The voltage-based determination module 630 is a module configured to make a determination of whether the portable auxiliary battery B is in good or bad condition by comparing a measured voltage value at an arbitrary (or a specific) SOC of the portable auxiliary battery B charged and discharged by the charging/discharging device 20 with a voltage value for each SOC stored in the database 70. In a state where the portable auxiliary battery B is charged or discharged to a predetermined level by the charging/discharging device 20, the controller 60 may obtain SOC information and voltage information of the portable auxiliary battery B through the connector 10. In addition, the database 70 may store a table having information of normal voltage values by manufacturer and SOC of the portable auxiliary battery B in normal condition. Accordingly, the voltage-based determination module 630 compares the voltage value at the arbitrary (or the specific) SOC of the portable auxiliary battery B with a reference voltage value at a corresponding SOC stored in the database 70, and determines that the portable auxiliary battery B is bad when the measured voltage value at that time exceeds a preset range compared to the reference voltage value at the corresponding SOC. For example, the voltage-based determination module 630 may determine that the portable auxiliary battery B is bad when the measured voltage value exceeds 5% of the reference voltage value at the corresponding SOC.

The resistance-based determination module 650 is a module configured to measure DC internal resistance values of the portable auxiliary battery B measured by the charging/discharging device 20 or the resistance-value measurement module 210 in two stages as a first resistance value and a second resistance value for respective SOCs different from each other, and make a determination of whether the portable auxiliary battery B is in good or bad condition by comparing the first resistance value and the second resistance value. In an ideal situation, resistance by SOC of the portable auxiliary battery B may have the same value. Accordingly, when the first resistance value and the second resistance value have a difference exceeding a preset range, the portable auxiliary battery B may be determined to be bad. For example, the resistance-based determination module 650 may measure a first resistance value at a time when the connected portable auxiliary battery B has an SOC of 60% and a second resistance value at a time when the connected portable auxiliary battery B has an SOC of 40%, and then make a determination of whether the portable auxiliary battery B is in good or bad condition by comparing the two resistance values, but the scope of the present disclosure is not limited by a numerical range described above.

The impedance-based determination module 670 is a module configured to make a determination of whether the portable auxiliary battery B is in good or bad condition by comparing a measured impedance at an arbitrary (or a specific) SOC of the portable auxiliary battery B charged and discharged by the charging/discharging device 10 with reference impedance for each SOC stored in the database 70. In a state where the portable auxiliary battery B is charged or discharged to a predetermined level by the charging/discharging device 20, the controller 60 may obtain SOC information of the portable auxiliary battery B through the connector 10. In addition, the controller 60 may obtain impedance information of the portable auxiliary battery B from the impedance measurement device 30. In addition, the database 70 may store a table having impedance information by manufacturer and SOC of the portable auxiliary battery B in normal condition. Accordingly, the impedance-based determination module 670 compares the measured impedance at the arbitrary (or the specific) SOC of the portable auxiliary battery B with the reference impedance at the corresponding SOC stored in the database 70, and makes the determination of whether the portable auxiliary battery B is in good or bad condition at that time when the measured impedance exceeds the preset range compared to the reference impedance at the corresponding SOC. For example, the impedance-based determination module 670 may make the determination of whether the portable auxiliary battery B is in good or bad condition when the measured impedance exceeds a range of about 6 to 8% on the basis of the reference impedance at the corresponding SOC.

The battery-condition score calculation module 690 is a module configured to calculate a condition score of the portable auxiliary battery B on the basis of the resistance value or impedance of the portable auxiliary battery B, the temperature value of the portable auxiliary battery B, and the information of released gas measured by the gas detection sensor 510. The condition score RiskScore of the portable auxiliary battery B may be calculated by using the following Equation (2).

RiskScore=α*ΔZ+β*Tg+γ*Gppm+δ*S (2)

In Equation (2), α, β, γ, and δ are weights, which may have preset values or variable values. In addition, ΔZ is a difference between impedance at an arbitrary (or a specific) SOC of the portable auxiliary battery B and impedance at a corresponding SOC stored in the database 70. Tg is a temperature of the portable auxiliary battery B, which may be a battery cell temperature at the time when the portable auxiliary battery B is connected to the connector 10, or may also be a battery cell temperature at an arbitrary (or a specific) point in time after the portable auxiliary battery B is connected to the connector 10. In addition, Gppm may have a value of “1” when gas released from the portable auxiliary battery B is detected through the gas detection sensor 510, and may have a value of “0” when the gas is not detected. Lastly, S is a value for any other arbitrary parameter. For example, S may have a value of “1” when a pressure value of the portable auxiliary battery B measured through the pressure sensor 530 changes by a predetermined level or more, and may have a value of “0” when the pressure value thereof changes by a level less than the predetermined level.

Referring to FIGS. 1 and 2, the database 70 is a component configured to store various kinds of information. For example, the database 70 may store a table having information of voltage values by manufacturer and SOC of the portable auxiliary battery B in normal condition. In addition, the database 70 may store a table having impedance information by manufacturer and SOC of the portable auxiliary battery B in normal condition. In addition, the database 70 may also store information on whether the portable auxiliary battery B is determined to be of good quality, which information is matched with a passenger's boarding pass information. To this end, a boarding pass reader 1d for allowing a boarding pass for each passenger to be inserted may be installed on one side of the housing part 1b of the fire prevention device 1.

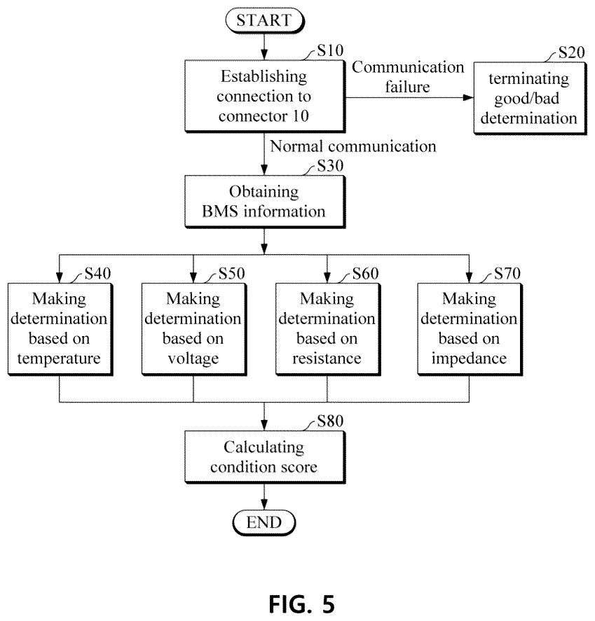

FIG. 5 is a flowchart of a fire prevention method for a multiparameter-based battery cell according to one exemplary embodiment of the present disclosure.

Hereinafter, the fire prevention method S1 for the multiparameter-based battery cell according to one exemplary embodiment of the present disclosure will be described in detail with reference to the attached drawings.

Referring to FIG. 5, first, in step S10, a connector 10 may be connected to a charging port P of a portable auxiliary battery B. In step S20, when a communication failure occurs despite the connector 10 being connected to the charging port P of the portable auxiliary battery B through step S10, determining whether the portable auxiliary battery B is in good or bad condition is terminated.

In step S30, when normal communication is established after performing step S10, a controller 60 obtains BMS information of the portable auxiliary battery B. It is preferable that the BMS information obtained by the controller 60 through step S30 is information on one or more of State Of Charge (SOC), cell voltage, cell temperature, internal resistance values, and the like of the portable auxiliary battery B, and is more preferable to be information including all kinds of the above information.

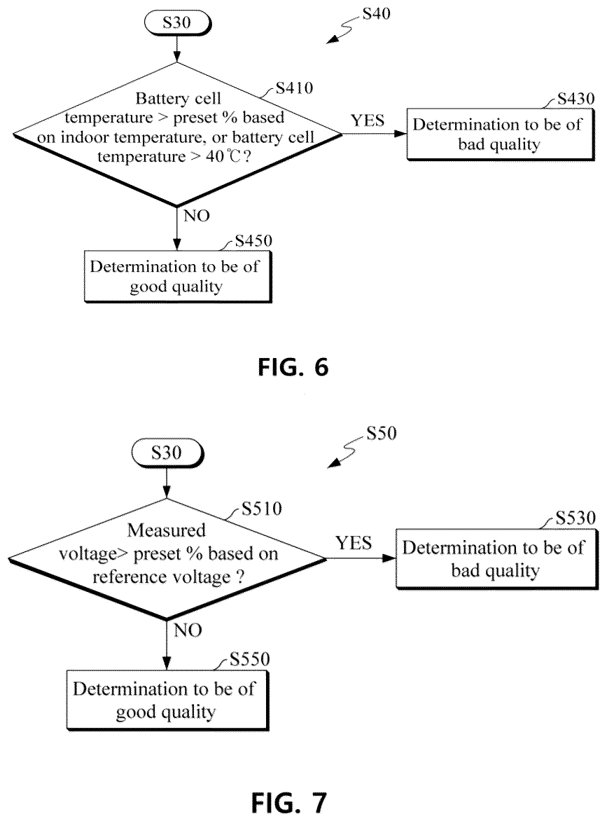

FIG. 6 is a flowchart of step S40 according to FIG. 5.

Referring to FIGS. 5 and 6, thereafter, in step S40, a determination of whether the portable auxiliary battery B is in good or bad condition may be made by comparing a battery cell temperature of the portable auxiliary battery B measured by a first temperature sensor B2 and an airport indoor temperature measured by a second temperature sensor 1c. To describe step S40 in detail, in step S410, this process makes the determination of whether the battery cell temperature of the portable auxiliary battery B exceeds a preset percentage based on the airport indoor temperature or exceeds a set value. Thereafter, in a case where the battery cell temperature exceeds the preset percentage based on the indoor temperature or when the battery cell temperature exceeds the preset value, the portable auxiliary battery B is determined to be bad in step S430, and when the opposite case is true, the portable auxiliary battery B is determined to be of good quality in step S450.

FIG. 7 is a flowchart of step S50 according to FIG. 5.

In addition, referring to FIGS. 5 and 7, in step S50, a determination of whether the portable auxiliary battery B is in good or bad condition may be made by comparing a measured voltage value at an arbitrary SOC of the portable auxiliary battery B charged and discharged by a charging/discharging device 20 with a reference voltage value for each SOC stored in a database 70. To describe step S50 in detail, first, in step S510, the voltage value at the arbitrary SOC of the portable auxiliary battery B is compared with the reference voltage value at a corresponding SOC stored in the database 70. Thereafter, in a case where the measured voltage value exceeds a preset range compared to the reference voltage value at the corresponding SOC, the portable auxiliary battery B is determined to be bad in step S530, and when the opposite case is true, the portable auxiliary battery B is determined to be of good quality in step S550.

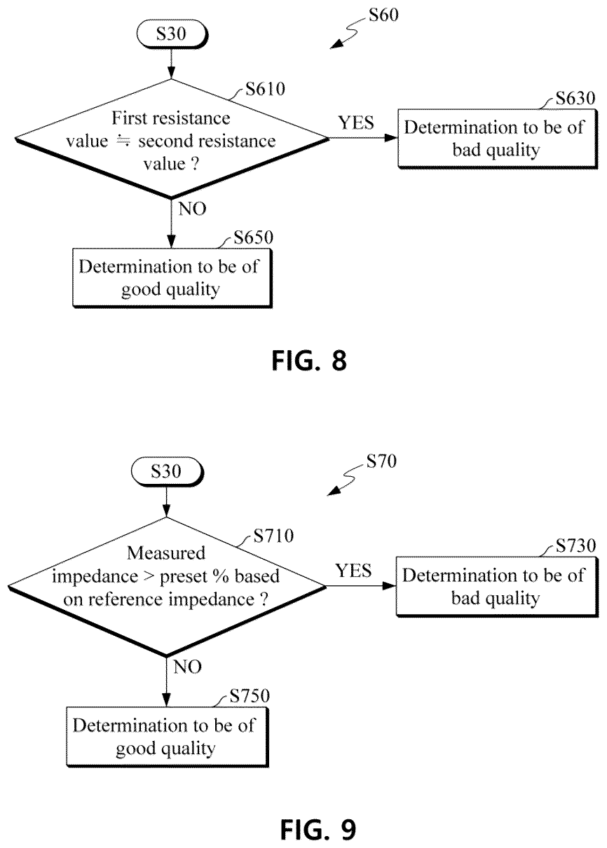

FIG. 8 is a flowchart of step S60 according to FIG. 5.

In addition, referring to FIGS. 5 and 8, in step S60, DC internal resistance values of the portable auxiliary battery B measured by the charging/discharging device 20 or the resistance-value measurement module 210 are measured in two stages as a first resistance value and a second resistance value for respective SOCs different from each other, and a determination of whether the portable auxiliary battery B is in good or bad condition may be made by comparing the first resistance value and the second resistance value. To describe step S60 in detail, first, in step S610, the resistance values of the portable auxiliary battery B are measured in two stages at SOCs different from each other. That is, the first resistance value and the second resistance value are measured. Then, in a case where a difference between the resistance values exceeds a preset range, the portable auxiliary battery B is determined to be bad in step S630, and when the opposite case is true, the portable auxiliary battery B is determined to be of good quality in step S650.

FIG. 9 is a flowchart of step S70 according to FIG. 5.

In addition, referring to FIGS. 5 and 9, a determination of whether the portable auxiliary battery B is in good or bad condition may be made by comparing a measured impedance at an arbitrary SOC of the portable auxiliary battery B charged and discharged by the charging/discharging device 10 with impedance for each SOC stored in the database 70. To describe step S70 in detail, first, in step S710, the impedance at the arbitrary SOC of the portable auxiliary battery B may be compared with the reference impedance at a corresponding SOC stored in the database 70. Then, in a case where the measured impedance exceeds a preset range compared to the reference impedance at the corresponding SOC, the portable auxiliary battery B may be determined to be bad in step S730, and when the opposite case is true, the portable auxiliary battery B may be determined to be of good quality in step S750.

In addition, any two steps arbitrarily selected from steps S40 to S70 described above may be performed substantially simultaneously, or may be performed in any order, and there is no separate limitation thereto.

Finally, referring to FIG. 5, in step S80, a condition score of the portable auxiliary battery B may be calculated on the basis of the resistance value or impedance of the portable auxiliary battery B, the temperature value of the portable auxiliary battery B, and the information of released gas measured by the gas detection sensor 510.

The detailed description above is illustrative of the present disclosure. In addition, the above description shows and describes preferred embodiments of the present disclosure, and the present disclosure may be used in various other combinations, modifications, and environments. That is, changes or modifications may be made within the scope of the concept of the embodiments disclosed in the present specification, the scope equivalent to the disclosed content described previously, and/or the scope of the skill or knowledge in the art. The above-described exemplary embodiments are to describe the best state for implementing the technical idea of the present disclosure, and various modifications required in the specific application fields and uses of the embodiment of the present disclosure are possible. Therefore, the detailed description of the embodiments of the present disclosure are not intended to limit the embodiments of the present disclosure to the disclosed embodiments.

What is claimed is:

1. A fire prevention device for a multiparameter-based battery cell, the fire prevention device comprising:
a connector connected to a charging port of a portable auxiliary battery;
a discharger for discharging the battery cell of the portable auxiliary battery through the connector;
an impedance measurement device for measuring internal impedance of the portable auxiliary battery connected through the connector;
a controller configured to obtain BMS information of the portable auxiliary battery and make a determination of whether the portable auxiliary battery is in good or bad condition on the basis of a plurality of parameters of voltage, resistance, and impedance; and
a database.

2. The fire prevention device of claim 1, wherein the discharger comprises:
a resistance-value measurement module configured to measure a direct current (DC) internal resistance value of the portable auxiliary battery.

3. The fire prevention device of claim 1, wherein the controller obtains a first temperature of the battery cell of the portable auxiliary battery measured through a first temperature sensor mounted on the portable auxiliary battery and a second temperature, which is an indoor temperature, measured through a second temperature sensor installed on one side of the fire prevention device, and
the controller comprises:
a temperature-based determination module configured to make the determination of whether the portable auxiliary battery is in the good or bad condition by comparing the first temperature and the second temperature.

4. The fire prevention device of claim 1, wherein the controller comprises:
a voltage-based determination module configured to make the determination of whether the portable auxiliary battery is in the good or bad condition by comparing a voltage value measured at an arbitrary State Of Charge (SOC) of the portable auxiliary battery discharged by the discharger with a voltage value for each SOC, stored in the database, of the portable auxiliary battery in normal condition.

5. The fire prevention device of claim 2, wherein the controller comprises:
a resistance-based determination module configured to make the determination of whether the portable auxiliary battery is in the good or bad condition through the DC internal resistance value of the portable auxiliary battery measured by the resistance-value measurement module.

6. The fire prevention device of claim 5, wherein the resistance-based determination module makes the determination of whether the portable auxiliary battery is in the good or bad condition through a first resistance value and a second resistance value, which are the portable auxiliary battery's DC internal resistance values for respective SOCs different from each other.

7. The fire prevention device of claim 1, wherein the controller comprises:
an impedance-based determination module configured to make the determination of whether the portable auxiliary battery is in the good or bad condition by comparing impedance measured at an arbitrary SOC of the portable auxiliary battery discharged by the discharger with impedance for each SOC, stored in the database, of the portable auxiliary battery in normal condition.

8. The fire prevention device of claim 1, wherein the controller comprises:
a battery-condition score calculation module configured to calculate a condition score of the portable auxiliary battery on the basis of a temperature of the portable auxiliary battery and a difference between impedance at a specific SOC of the portable auxiliary battery and impedance at a corresponding SOC stored in the database.

9. The fire prevention device of claim 1, further comprising:
a printing module configured to print a barcode or a QR code containing information of the good/bad determination on a printed material to be provided to a user after making the determination of whether the portable auxiliary battery is in the good or bad condition through the controller.

10. The fire prevention device of claim 1, further comprising:
an airtight part configured to allow the portable auxiliary battery connected to the connector to be received.

11. The fire prevention device of claim 10, wherein the airtight part comprises:
a gas detection sensor installed on one side thereof and configured to detect gas released from the portable auxiliary battery into an internal space of the airtight part.

12. The fire prevention device of claim 11, wherein the airtight part further comprises:
a pressure sensor installed on one side thereof and configured to detect a pressure value during a discharging process of the portable auxiliary battery.

13. The fire prevention device of claim 1, wherein the database stores information of the good/bad determination of the portable auxiliary battery, the information being determined by the controller and matched with a passenger's boarding pass information.

14. The fire prevention device of claim 13, further comprising:
a housing part forming an exterior of the fire prevention device; and
a boarding pass reader installed on one side of the housing part and to which a boarding pass for each passenger is allowed to be inserted.

15. The fire prevention device of claim 12, wherein the airtight part further comprises:
an automatic fire extinguishing part configured to automatically extinguish a fire occurring in the internal space of the airtight part.

16. The fire prevention device of claim 15, wherein the airtight part further comprises:
a gas suction part configured to allow gas released outside a casing of the portable auxiliary battery during a charging and discharging process of the portable auxiliary battery to be released outside the airtight part.
